(19) United States
(12) Patent Application Publication
(10) Pub. No.: US 2005/0114892 A1
(43) Pub. Date: May 26, 2005

(54) Low orbit satellite communication with mobile medical equipment incorporating global positioning system

(76) Inventor: Peter Chan, Harland, WI (US)

Correspondence Address: Patrick S. Yoder;FLETCHER YODER, P.O. Box 692289, Houston, TX, 77269-2289, US

(21) Appl. No.: 10/723,865

(22) Filed: Nov. 26, 2003

(51) Int. Cl. H04N 7/18; H04N 7/20; A61B 5/05

(52) U.S. Cl. 725/63; 725/67; 600/410; 725/75

(57) ABSTRACT

A technique is provided for communicating monitored data along with position data for an imaging system to a service center. In the present technique, an imaging system may be monitored to measure the operational conditions of the imaging system. In addition, positioning signals may be received at the imaging system from satellites associated with a satellite based positioning system to determine the position of the imaging system relative to a location on a map. With the operational conditions and the position data, the imaging system may utilize a low earth orbit transceiver to transmit the data to the service center.

### BACKGROUND OF THE INVENTION

The present invention relates generally to medical equipment. More particularly, the invention relates to a technique for exchanging information between medical equipment and a service center via a low earth orbit satellite communication system.

A wide variety of services and procedures are utilized by medical personnel to meet the needs of their patients. Typically, medical practitioners, such as physicians, employ medical imaging systems to diagnose patients. The imaging systems may include magnetic resonance imaging (MRI) systems, computed tomography (CT) systems, ultrasound systems, x-ray systems, and so forth. The imaging systems may produce detailed images of a patient's internal tissues and organs, thereby mitigating the need for invasive exploratory procedures and providing valuable tools for identifying and diagnosing disease and for verifying wellness.

As the imaging systems are omnipresent in typical medical environments, the imaging systems may be dispersed in a variety of geographical locations to provide the medical services and equipment to patients. Some of the geographic locations may include remote locations or mobile environments that present problems with service or support because of limited communication capabilities. For instance, the imaging system may be located in a rural hospital that does not have physical connections to a network or in a mobile environment that is moved from one location to another. In these environments, the imaging system may not be able to communicate with a radiology department information system (RIS), a hospital information system (HIS), or other control systems by conventional communications systems to coordinate the operation of the imaging system. In addition, radiologists, diagnosing physicians, and vendor support organizations may not be able to communicate with the imaging system, as well. As such, the geographical location of the medical imaging system may present certain obstacles for maintaining communication with others systems or personnel.

For example, if the imaging system is an MRI system, then certain operational conditions may have to be maintained for the MRI system. Typically, an MRI system includes super-conductive electromagnets that may be continuously bathed in a cryogen, at temperatures near absolute zero—approximately −271 C or 4 K. The MRI system may monitor the cryogenic liquids because the cryogenic liquids are relatively expensive to produce and maintain. The vendor support organization may communicate with the MRI system to maintain the operation of the MRI system and monitor potential situations based on data received from sensors and monitors. However, in the mobile or rural environment, communication with the MRI system may be an obstacle or may not be possible. As a result, the MRI system may experience problems or failures that may be avoided by proper communication of the monitored conditions.

Additionally, because the imaging system may be utilized in a mobile environment, the scheduling of field service technicians is difficult and often expensive. With the mobile imaging system, the service technician may have to travel to the imaging system's location to perform a specific service. Typically, the field service engineer may coordinate the imaging system's service with the scheduled operation of the imaging system. However, if the imaging system's schedule is incorrect or changes have not been incorporated into its schedule, then the field service engineer may waste time in traveling to an incorrect location or may not be able to address the maintenance needs of the imaging system. Thus, support of the imaging system may be decreased because of the inability to determine the location of the mobile imaging system, or to collect data needed to determine the operational state of service needs of the system.

As a result, there is a particular need at present for a technique which would permit and coordinate the exchange of information between an imaging system and service center. The need extends both to imaging systems at remote locations as well as in the mobile environments. Because external resources may support the imaging system, communication with those resources may be utilized to enhance the operation of the imaging system under the present techniques. Further, a need exists for a mobile imaging system to provide location information for coordinating service calls and support materials.

### BRIEF DESCRIPTION OF THE INVENTION

The present invention relates generally to providing monitored data and position information for an imaging system to a service center to support the imaging system. In particular, the technique provides for communicating the monitored data and position information between the imaging system and the service center via a low earth orbit satellite system. In addition, the position information may be determined from position signals received from a global positioning system. In this manner, monitored data and position information associated with the imaging system may be provided to the service center to enhance the operation of the imaging system. Similarly, the monitored data and position information may reduce the costs of supporting the imaging system.

In accordance with one aspect of the present technique, a method for communicating with an imaging system is provided. An imaging system may be monitored for operational conditions associated with the imaging system. The imaging system may also receive position signals from the satellites. Then, the imaging system may determine the location of the imaging system based on the position signals. Then, the imaging system may transmit the operational data along with the location of the imaging system to a service center via a low earth orbit system. Systems and computer programs that afford functionality of the type defined by this method are also provided by the present technique.

### BRIEF DESCRIPTION OF THE DRAWINGS

FIG. 1 is a diagrammatical overview of a communication system for communicating information from an imaging system to one or more service centers in accordance with certain aspects of the present technique;

FIG. 2 is a diagrammatical view of an exemplary mobile imaging system in accordance with certain aspects of the present technique for use in the system shown in FIG. 1;

FIG. 3 is a diagrammatical representation of an exemplary mobile imaging communication system having features in accordance with the present technique; and

FIG. 4 is a flow chart of exemplary logic for exchanging data in accordance with the present technique.

### DETAILED DESCRIPTION OF SPECIFIC EMBODIMENTS

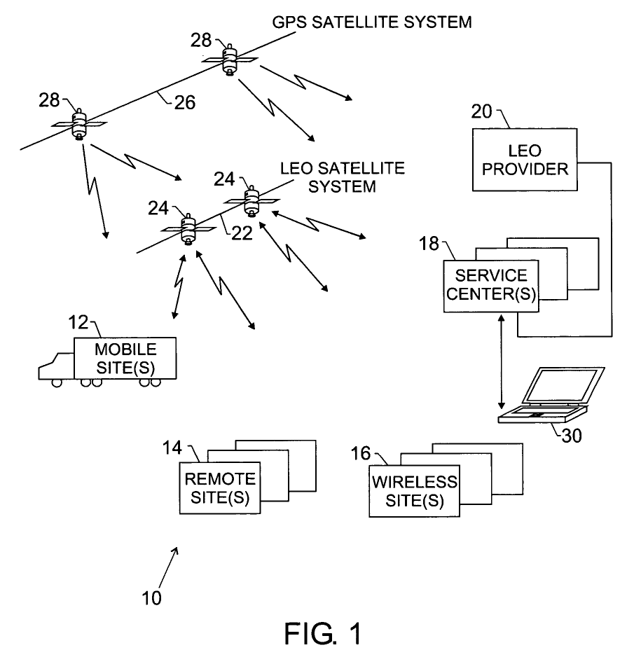

Turning now to the drawings, and referring initially to FIG. 1, a communication system, designated generally by reference number 10, is illustrated for transmitting operational data or conditions between a medical imaging system and a service center. The communication system 10 may permit the exchange of data, such as remote monitoring data, operational data, location information, and positioning information. As illustrated in FIG. 1, the communication system 10 generally includes one or more mobile imaging sites 12, one or more remote sites 14, and/or one or more wireless sites 16, which communicate with one or more remote service centers 18 via a low earth orbit (LEO) satellite system 22, as discussed below. Also, the sites 12-16 may receive positioning signals from a global positioning system (GPS) satellite system 26 to supply the position of the sites 12-16.

The mobile sites 12, the remote sites 14, and the wireless sites 16 may utilize the LEO satellite system 22 to communicate with the data service centers 18. Each of these sites 12, 14, and 16 may include imaging systems and other associated hardware, which are discussed below. The mobile sites 12 may be one or more specialized diagnostic treatment facilities that may be moved from one location to another to provide medical services. The remote sites 14 may be a fixed structure or structures, such as office buildings or other such sites, which are located in a geographically remote area. The remote sites 14 also may be rural hospitals or clinics that are geographically dispersed to provide medical services. Similar to the mobile sites 12 and the remote sites 14, the wireless sites 16 may be specialized diagnostic treatment facilities that may be semi-permanent structures that may be moved from one location to another.

In general, the service centers 18 may include facilities for processing data and requests from the sites 12-16. The service centers 18 may be vendor support organizations or service providers that provide maintenance support, such as repairs and servicing of the imaging system or other hardware at the sites 12-16. The service centers 18 may monitor the operational conditions of the imaging system based on a subscription or contract basis. The data and requests exchanged between the sites 12-16 and the service centers 18 may include monitored data, operational data, location data, scheduling data, or other information that may be provided to the service centers 18 in accordance with the techniques described below.

The service centers 18 may also be in communication with various support engineers and technicians, such as field service engineers 30, to provide service to the sites 12-16. Particularly, the service centers 18 may communicate with the field service engineers 30 to coordinate various support activities for the imaging systems at the sites 12-16. For instance, the field service engineers 30 may be utilized by service centers 18 to travel to the sites 12-16 to perform maintenance or troubleshoot problems on different imaging systems. Also, the field service engineers 30 may be associated with vehicles that supply parts or materials to the imaging systems at the sites 12-16.

The LEO provider 20 may also communicate with the service centers 18 via a network link. The network link may be via a terrestrial link, such as a private circuit, a virtual private network connection, an Internet connection, or other suitable type of connections. The LEO provider 20 may act as a gateway earth station, a gateway control center, or a network control center, which is utilized to communicate with the LEO satellite system 22. The interaction of the LEO provider 20 is further discussed below in greater detail.

To exchange data between the sites 12-16, the service centers 18, and the field service engineers 30, the LEO satellite system 22 may be utilized to provide connectivity between the various systems. Generally, the LEO satellite system 22 is a high-capacity broadband satellite network that provides global coverage with lower latency than higher orbit satellites. The LEO satellite system 22 may include one or more LEO satellites 24 that orbit within a range of 100 to 1000 miles above the Earth. The LEO satellite system 22 may utilize communication schemes and protocols, such as very high frequency (VHF), ultra high frequency (UHF), microwave, Time Division for Multiple Access (TDMA), Code Division Multiple Access (CDMA), Frequency Division Multiple Access (FDMA), radio frequencies (RF), and/or any other suitable frequency bands, to establish the network links.

To provide connectivity, the LEO satellites 24 may exchange data with ground units, such as subscriber communicators or terminal units, that may be located at the sites 12-16, service centers 18, and/or LEO provider 20. These ground units may utilize wireless technologies to establish communication links between the different locations. Also, the LEO satellite system 22 may utilize various protocols to route the packets from one location to another. These protocols may be proprietary or may be public protocols, such as Internet protocol (IP). As an example of the use of the LEO satellites 24, one of the mobile sites 12 may send data, such as operational data, to one of the service centers 18 via one of the LEO satellites 24. The LEO satellite may act as a conduit that transmits the data between the mobile site and the service center. Regardless of the location of the mobile site and the service center, the mobile site may provide operational data to the service center through this LEO satellite link. In addition, the LEO satellites 24 may route the data between different LEO satellites 24 to exchange the monitored data between the service centers 18 and one of the mobile sites 12.

As another communication path, the sites 12-16 may communicate with the service centers 18 via the LEO provider 20. As discussed above, the LEO provider 20 may have a terrestrial link to the service centers 18. This network link provides the sites 12-16 with an additional path from the LEO satellites 24 to the service centers 18 through the LEO provider 20. For instance, one of the mobile sites 12 may transmit data to one of the LEO satellites 24. The data may be forwarded through the LEO provider 20 to one of the service centers 18. In this manner, the LEO provider 20 may provide an additional network path for the LEO satellite system 22.

Regardless of the path utilized, the LEO satellite system 22 provides communication links to each of the sites 12-16, which may not be accessible by physical communication lines or viable for other technologies. For instance, cellular technologies have a more limited coverage area than the LEO satellite system 22 because the height of the tower limits the range of coverage. As a result, the LEO satellite system 22 beneficially provides the sites 12-16 with an enhanced coverage area because the LEO satellite system 22 orbits the Earth, which provides a larger coverage area. This allows the service centers 18 to utilize a single communication system to provide coverage to sites 12-16, which may be geographically dispersed.

Furthermore, while geo-synchronous (GEO) satellites provide coverage to large areas, the high equipment costs and longer communication delays present problems that are detrimental to communication systems. As a result, the LEO satellite system 22 beneficially provides the sites 12-16 with a responsive monitoring system that does not experience the longer delays of the higher orbit GEO satellites. Also, the LEO satellite system 22 is a more cost effective system as compared to GEO satellite systems. As a result, the LEO satellite system 22 provides a cost effective communication system, which is able to provide communication for remote and mobile locations.

In addition to the LEO satellite system 22, the GPS system 26 may be utilized to provide location information for mobile sites 12, remote sites 14, wireless sites 16, and/or the field service engineers 30. It should be noted that the GPS system 26 may be any of a variety of satellite based positioning system, but is simply used for illustrative purposes. The GPS system 26 may include one or more GPS satellites 28 to provide position data to a receiver associated with the mobile sites 12, the remote sites 14, the wireless sites 16, and/or the field service engineers 30. Through the use of position signals, the GPS system 26 may provide the sites 12-16 and field service engineers 30 with position data or information that corresponds to a location on a map.

For instance, the GPS system 26 may include GPS satellites 28 which are utilized to provide position data. The GPS satellites 28 may each broadcast a signal containing the specific location of the satellite at a specific time. The GPS receiver, which is associated with the sites 12-16 or field service engineers 30, may receive the signals from the GPS satellites 28. The determination of the receiver's location may depend on the receipt of signals from 3 or more of the GPS satellites 28. With these signals, the GPS receiver may use triangulation to determine the location of the sites 12-16 or field service engineers 30, which is associated with the GPS receiver. As a result, the sites 12-16 and the field service engineers 30 may provide location or position data to the service centers 18 via the LEO satellite system 22.

Through the use of the GPS system 26, the equipment at sites 12-16 or the field service engineers 30 may determine the location of the sites 12-16 and the field service engineers 30 at a specific time. This is beneficial because the service centers 18 may be notified of the location of the field service engineers 30 and the location of the imaging system associated with one of the sites 12-16. This allows the service centers 18 to better coordinate field service engineers 30 that support the sites 12-16 to optimize the time and travel of the field service engineers 30. In addition, by determining the location of the imaging system in the sites 12-16, the service centers 18 may coordinate the supply of parts and material to the imaging system at the sites 12-16.

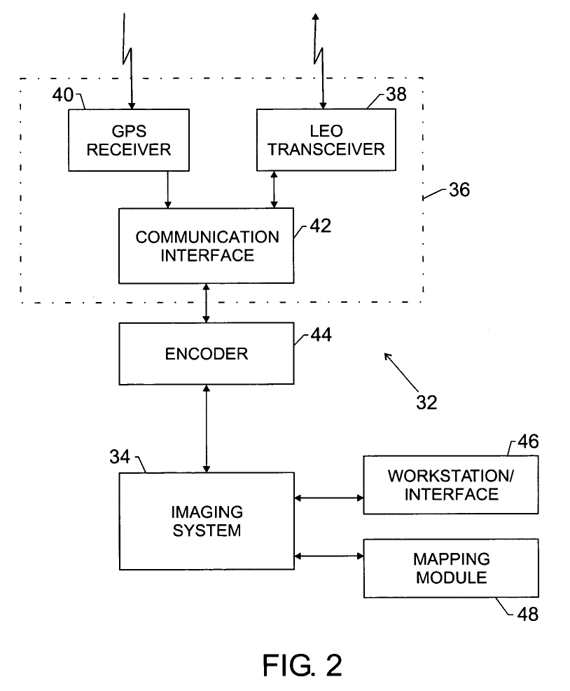

As a more specific example of the sites 12-16, FIG. 2 is an illustration of a diagrammatical view of an exemplary site in accordance with certain aspects of the present technique of the system shown in FIG. 1. The imaging site 32, which may be one of the mobile sites 12, one of the remote sites 14, or one of the wireless sites 16 (FIG. 1), may include an imaging system 34 that is connected to a communication module 36 through an encoder 44 to communicate with other systems or facilities via the LEO satellite system 22. The other systems and facilities may include other sites 12-16, the service centers 18, and/or field service engineers 30 (FIG. 1). In addition, the position of the imaging system 34 may be ascertained from the GPS system 26, as discussed above.

The imaging site 32 may utilize the communication module 36 to communicate with the external devices or systems. The communication module 36 may be a ground unit, such as a subscriber communicator or a terminal unit, which is utilized to establish communication links with other devices or system through satellites, such as the LEO satellites 24 and the GPS satellites 28 (FIG. 1). The ground unit may utilize different wireless technologies to establish the communication links. Also, the communication module 36 may utilize various protocols to route or guide packets from the external devices to the components or systems within the imaging site 32, which may include the imaging system 34 or specific monitors associated with the imaging system 34. The protocols may be proprietary or public protocols, such as Internet protocol (IP), for example.

The communication module 36 may include a LEO transceiver 38, a GPS receiver 40, and a communication interface 42. The LEO transceiver 38 may be a device that transmits and receives analog or digital signals over wireless links. For instance, the LEO transceiver 38 may transmit operational data to the service center and receive command data, such as instructions and setting information, from the service center. The LEO transceiver 38 may be any suitable type of modem, such as a microwave, UHF, or VHF modem, that is used to transmit and receive messages. The GPS receiver 40 may receive signals from satellites, such as the GPS satellites 28 (FIG. 1). As discussed above, the GPS receiver 40 may determine the location of the imaging system 34 from the received position signals. The communication interface 42 may be utilized to exchange messages between the LEO transceiver 38, the GPS receiver 40, and external systems in communication with the communication module 36. The communication interface 42 may modify the format of information in the messages to a format that is acceptable to other devices within the imaging site 32. The communication interface 42 may also map data packets between the LEO transceiver 38, GPS receiver 40, and external devices through a specific port configuration, which may include RS-232C or Ethernet.

To provide data to external systems, the communication interface 42 of the communication module 36 may interact with an encoder 44 that is coupled to the imaging system 34. The encoder 44 may utilize different algorithms or encoding techniques to compress or encode data being transmitted to the communication module 36. Likewise, the encoder 44 may decrypt or uncompress data received from the communication interface 42. To encode/decode the data, the encoder 44 may utilize hexadecimal formats or Huffman encoding to reduce the amount of data being transmitted over the satellite links. For instance, the encoder 44 may receive raw monitored data from the imaging system 34. The encoder 44 may modify the raw monitored data into a hexadecimal format for transmission through the communication module 36. The hexadecimal format may utilize less bandwidth than the raw data. Similarly, the encoder may convert data from hexadecimal format into a format that is utilized by the components of the imaging system 34.

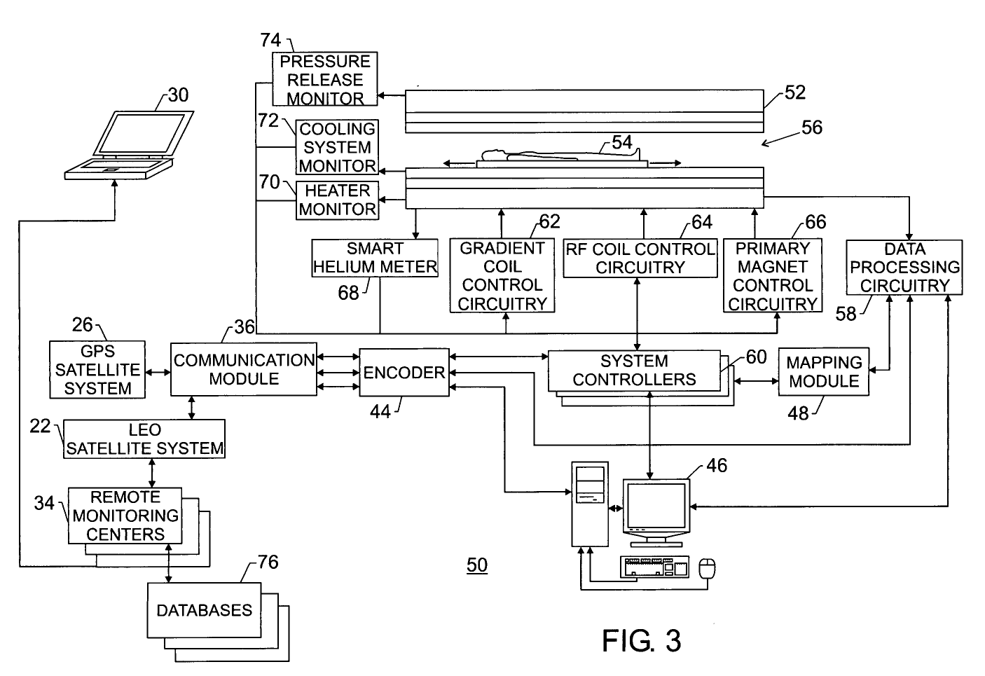

The imaging system 34 may be a medical diagnostic imaging system designed to produce useful images of patient's anatomies in accordance with particular physics or modalities. For instance, the imaging system 34 may be a magnetic resonance imaging (MRI) system, computed tomography (CT) system, ultrasound system, x-ray system, nuclear magnetic resonance (NMR) system, or other suitable imaging device. The imaging system 34 may also include patient monitors, sensors, transducers, imaging monitors, and other signal generating or feedback devices, which is further discussed below in an exemplary imaging system of FIG. 3.

The imaging system 34 may be coupled to a workstation/interface 46 and a mapping module 48 to provide access to and the location of the imaging system 34. The workstation/interface 46 may be a computer system utilized to interface with the imaging system 34. The workstation/interface 46 may include a computer system with a keyboard, a monitor, and a mouse, which are utilized to enter data into and display data from the imaging system 34. The mapping module 48 may be utilized to determine the location or position of the imaging system 34. The mapping module 48 may include electronic maps or other guidance tools used to aid in determining the location of the imaging system 34. For instance, the mapping module 48 may utilize the position signals received from the GPS receiver 40 to calculate the position of the imaging system 32. The mapping module 48 may then compare the calculated position with a known map stored in the mapping module 48 to determine the position of the imaging system 32.

Beneficially, the use of the communication module 36 provides the imaging site 32 with an ability to communicate with external locations that may be geographically dispersed. As a result, the imaging system 34 may provide the location along with data relating to the operation of the imaging system 32 to the external systems to enhance the support of the imaging system. As shown in FIG. 3, an exemplary mobile imaging communication system 50, which may be located in one of the mobile sites 18 (FIG. 1), is illustrated. Although the present technique is described with respect to a MRI system, it should be noted that the present technique may be applied to any number of imaging systems or devices, as discussed above. The exemplary mobile imaging communication system 50 includes an exemplary MRI scanner 52 along with various monitors and sensors, such as a smart helium meter 68, a heater monitor 70, a cooling system monitor 72, and a pressure release monitor 74. Certain details relating to the structure and operation of the exemplary MRI system are provided below for a better understanding of the types of data that can be transmitted, monitored and even controlled via the links and techniques described herein.

To obtain diagnostic images of a patient 54, a medical professional may direct the patient 54 into a patient bore 56 of the MRI scanner 52. A main magnetic field (i.e., 0.5-2.0 Tesla) is generally present in the patient bore 56. This field is produced by a super-conductive electromagnet disposed circumferentially about the patient bore 56. The super-conductive electromagnet is maintained at super-conducting temperatures (e.g., 1-5 degrees Kelvin) to reduce the electrical resistance in the magnet coils to substantially zero. The super-conductive nature of the electromagnet reduces the electrical requirements for producing the magnetic field, thereby making the MRI scanner 52 more economical to operate. To manipulate the main magnetic field and to obtain diagnostic images, the MRI scanner 52 includes gradient magnets or coils, and radio frequency (RF) coils (not shown), both of which may be of generally known construction.

The MRI scanner 52 may interact with any number of control and monitoring circuits to perform the imaging functions. For instance, the MRI scanner 52 is coupled to data processing circuitry 58, which receives the detected imaging signals and processes the signals to obtain data for image reconstruction. In typical MRI scanners 52, the data processing circuitry 58 digitizes the received signals and performs a two-dimensional fast Fourier transform on the signals to decode specific locations in the selected slice from which the received signals originated, thereby producing image data representative of the patient's internal tissue and organs, or more generally, features of interest of a subject. The resulting image data may be forwarded to the workstation/interface 46 for viewing. The image data may also be sent to a remote data repository for storage. Advantageously, the data processing circuitry 58 may perform a wide range of other functions, such as image enhancement, dynamic range adjustment, intensity adjustment, smoothing, sharpening, and so forth. However, it should be appreciated that such functions may also be performed by software and/or hardware included in the workstation/interface 46 as well as at remote locations, such as one of the service centers 18.

Additionally, certain control and monitoring circuits may function under the direction of one or more system controllers 60, such as a heater controller and a cooling system controller. The system controllers 60 may permit some amount of adaptation or configuration of the examination sequence by means of the workstation/interface 46. The workstation/interface 46 may provide a graphical user interface (GUI) to an individual for the receipt of information from and the input of commands to the MRI scanner 52.

The control circuits that interact with the system controllers 60 may be utilized to operate the coils and magnets of the MRI system 52. By way of example, the gradient coils, the RF coils, and the main magnet may be controlled by gradient coil control circuitry 62, RF coil control circuitry 64, and main magnet control circuitry 66, respectively. These different control circuits may be utilized to create and adjust the magnetic fields within the patient bore 56.

Moreover, as discussed below, various monitors may communicate with the system controllers 60 to verify the operational and measured conditions of the MRI scanner 52. For instance, the MRI scanner 52 may be bathed in a cryogen, such as liquid helium, which is circulated around the patient bore 56 and electromagnet (not shown). The liquid helium cools the electromagnet to super-conductive temperatures (e.g., −271 C or 4 k) to reduce the electrical resistance, which reduces the electrical loads of the MRI scanner 52. Because the liquid helium vaporizes into a gaseous state (i.e., gaseous helium) at relatively low temperatures, a heating system and a cooling system may be used to adjust the temperature within the MRI scanner 52. The adjustment of the temperature may recondense gaseous helium back into liquid helium to recycle the liquid helium. As the temperature changes, the pressure within the MRI scanner 52 may change as well. When the pressure exceeds a certain threshold, such as 4 psi, a vent within the MRI scanner 52 may release excess gaseous helium. However, because helium is relatively expensive, the venting of the helium is to be avoided.

To maintain the operation of the MRI scanner 52, monitors may be used to provide operational data to the service centers 18 that provide support and maintenance for the MRI scanner 52. The monitors may include the smart helium meter 68, the heater monitor 70, the cooling system monitor 72, and the pressure release monitor 74. The smart helium meter 68 may measure the level of liquid or gaseous helium along with the pressure within the MRI scanner 52. The heater monitor 70 may measure the heater duty cycle or duration of time that the heater in the MRI scanner 52 is operating. The activity of the heater may indicate that the heater is about to fail or not functioning properly. The cooling system monitor 72 may measure the duration of time that the chiller or compressor is being utilized. The increased activity of the cooling system may indicate that the MRI scanner 52 is not cooling enough to recondense the gaseous helium. The pressure release monitor 74 may measure the release of any gaseous helium along with the pressure within the MRI scanner 52. The pressure release monitor 74 may be utilized to determine the amount of liquid helium within the MRI scanner 52. The monitored data from each of these monitors 68-74 may be provided to the system controllers 60, which may communicate the monitored data with external vendors or service centers 18.

To provide remote support and maintenance for the MRI scanner 52, the MRI scanner 52 may communicate with remote locations and devices via communication module 36. The communication module 36 may exchange data between components associated with the MRI scanner 52 and the service centers 18 via the LEO satellite system 22. For instance, the service centers 18 may communicate commands to the MRI scanner 52 from the service center. As discussed above, the service centers 18 may interact with the MRI scanner 52 to monitor and adjust the operating parameters or conditions of the MRI scanner 52 remotely. The service centers 18 may include one or more databases 76, which may store large volumes of image data, operating data, monitored data, scheduling data, and position data from the monitors 68-74 of associated with the MRI scanner 52 along with other mobile imaging systems. That is, data from multiple MRI scanners, other imaging systems, and/or patients may be stored in a central location.

In certain instances, field technicians at the service centers 18 or field service engineers 30 may access data or operating conditions from the system controllers 60 or monitors 68-74 associated with the MRI scanner 52. For instance, with the equilibrium in the MRI scanner 52 being biased toward the gaseous phase, the smart helium meter 68 may indicate that the level of liquid helium is reaching a low level. At a low level of liquid helium, the MRI scanner 52 may lose super-conductivity and cease to operate. With the communication module 36 and mapping module 48, the mobile imaging communication system 50 is able to determine the position of the MRI scanner 52 in relation to a location on a map. The position data along with the monitored data from the monitors 68-74 may be combined as operational data, which is transmitted to the service centers 18 via the LEO satellite system 22. As a result, an operator at one of the service centers 18 may be notified of the problem with the low level of liquid helium from the operational data. The operator may dispatch a vehicle to the MRI scanner 52 to refill the liquid helium prior to the MRI scanner 52 failing because the operator is able to determine the location of and problem with the MRI scanner 52. As a result, the expense of cooling the MRI scanner 52 back to super-conductive temperatures may be prevented by the proactive maintenance of the MRI scanner 52.

Alternatively, the cooling system monitor 72 may indicate that the compressor is operating for extended periods of time. This may indicate that the cooling system is experiencing a problem in condensing the gaseous helium into liquid form. The position data along with the monitored data from the cooling system monitor 72 may be transmitted to the service centers 18 via the LEO satellite system 22. An operator at one of the service centers 18 may compare the monitored data from the cooling system monitor 72 with previously stored data in one of the databases 36. The operator may determine that the components of the cooling system may need repair and prepare an appropriate response. Accordingly, the operator may dispatch one of the field service engineers 30 to perform maintenance on the MRI scanner 52, or may schedule the MRI scanner 52 for maintenance based on the operational schedule of the MRI scanner 52. With either response, the MRI scanner 52 may be repaired without impacting the operational schedule of the MRI scanner 52.

Beneficially, by utilizing the monitored data along with the position data, the MRI scanner 52 may remain operational for longer periods of time. For instance, the personnel at the service centers 18 may determine the location and operational conditions that relate to the MRI scanner 52, which may be utilized to track the performance of the MRI scanner 52. This information may reduce the loss of cryogens by providing the service centers 18 with monitored data that relates to the cryogen levels, regardless of the location of the MRI scanner 52. As a result, the cryogens may be refilled before the MRI scanner 52 loses super-conductivity. Also, the information allows the personnel at the service centers 18 to coordinate the maintenance and part replacement of the MRI scanner 52 based on the planned operational schedule of the MRI scanner 52. As a result, the MRI scanner 52 may remain operational for longer periods of time by reducing the number unexpected breakdowns.

Additionally, the monitored data along with the position data may be utilized to reduce the costs associated with supporting the MRI scanner 52. By knowing the location of the MRI scanner 52, the field service engineers 30 may reduce the time and costs associated with traveling to the MRI scanner 52. In addition, the field service engineers 30 may know the expected problems that the MRI scanner 52 is experiencing from the monitored data and position data transmitted to the service centers 18. Accordingly, the personnel at the service centers 18 may provide materials and parts from convenient locations to reduce costs and delays in providing the support of the MRI scanner 52. For instance, if the cryogen level is low, the personnel at the service centers 18 may direct a vehicle to refill the cryogen from a location that is close to the MRI scanner 52. As a result, the MRI scanner 52 may be operational for longer periods with minimal disruption due to service or maintenance, while the support costs from vendors may also be reduced because the location of the MRI scanner 52 is known.

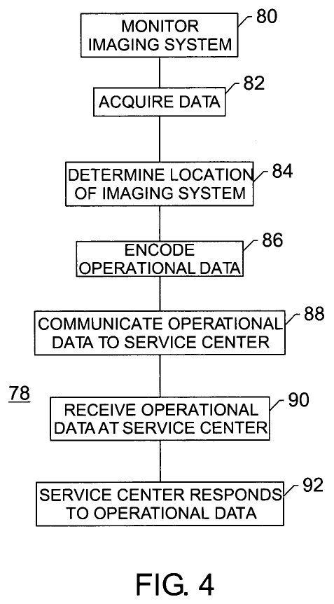

To further understand the exchange of data in the mobile imaging communication system 50, FIG. 4 illustrates a flow chart of exemplary logic for exchanging data, such as monitored data and position data, between the service center and the imaging system in accordance with the present technique. In the flow chart, which is generally referred to by reference numeral 78, a monitor, such as the one of the monitors 68-74 (FIG. 3), may provide monitored data to a service center, such as one of the service centers 18 (FIG. 3). In addition, location information that relates to the imaging system may be provided to the service center for enhancing support or services provided to the imaging system. With the monitored data and position data, the service center may determine the operational condition and location of the imaging system to allocate the appropriate resources to enhance the support of the imaging system.

The flow chart 78 begins with the collection of data and information in blocks 80-84. At block 80, a monitor may be used to measure the imaging system for monitored data and operational conditions. At block 82, the system controller, which may be one of the system controllers 60 (FIG. 3), may acquire the measured data from the monitor. Then, the position of the imaging system may be determined, as shown in block 84. The determination of the position of the imaging system may be based on the positioning signals received by the GPS receiver 40 (FIG. 2) in the communication module 36 (FIGS. 2-3). The positioning signals may utilize the mapping module 48 (FIGS. 2-3) to relate the imaging system's position to a location on a map, as discussed above.

Once the operational data is collected, the imaging system may communicate the operational data to the service center, as shown in blocks 86-92. In block 86, the imaging system may encode the monitored data and the position data as operational data. The operational data may be encoded by an encoder, such as encoder 44 (FIG. 3), into various formats, as discussed above. Then, the operational data may be communicated to the service center in block 88. The operational data may be transmitted through the communication module 36 via the LEO satellite system 22 (FIG. 3). The operational data may be received at the service center, as shown in block 90. At block 92, the service center may respond to the operational data. The response by the service center may include storing the operational data in a database, which may be one of the databases 76 (FIG. 3). Also, the response may include an operator at the service center analyzing operational data and dispatching a field service engineer, directing a vehicle with materials to the imaging system, or scheduling maintenance of the imaging system.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A medical communications system, the system comprising:
an imaging system;
a low earth orbit transceiver coupled to the imaging system, wherein the low earth orbit transceiver receives command data and transmits operational data to a service center; and
a satellite based positioning system receiver coupled to the imaging system, wherein the satellite based positioning system receiver obtains a plurality of position signals from a plurality of position satellites to determine a position of the imaging system.

2. The system of claim 1, wherein the imaging system is located in a vehicle.

3. The system of claim 1, wherein the imaging system comprises at least one monitor for monitoring at least one operational condition of the imaging system and providing at least one operational condition to the service center.

4. The system of claim 3, wherein the imaging system comprises a magnetic resonance imaging system.

5. The system of claim 4, wherein the at least one monitor comprises a smart helium meter.

6. The system of claim 4, wherein the at least one monitor comprises a plurality of sensors that measure a level of liquid helium within the magnetic resonance imaging system.

7. The system of claim 3, wherein the at least one monitor comprises a cooling system monitor.

8. The system of claim 3, wherein the imaging system comprises a computed tomography system.

9. The system of claim 3, wherein the operational data comprises position data and at least one operational condition.

10. The system of claim 1, wherein the satellite based positioning system receiver comprises a global positioning system receiver.

11. A method for communicating data with a medical imaging system, the method comprising the steps of:
monitoring an imaging system for monitored data;
receiving a plurality of position signals from a plurality of satellites;
determining location information of the imaging system from the plurality of position signals; and
transmitting operational data from the imaging system to at least one service center via a low earth orbit satellite system, wherein the operational data comprises the monitored data and the location information.

12. The method of claim 11, comprising the further step of the at least one service center responding to the operational data.

13. The method of claim 11, comprising the further step of acquiring monitored data from at least one monitor coupled to the imaging system.

14. The method of claim 11, comprising the further step of polling the at least one monitor coupled to the imaging system.

15. The method of claim 14, wherein the imaging system comprises a magnetic resonance imaging system.

16. The method of claim 15, wherein the at least one monitor comprises a smart helium meter.

17. The method of claim 11, comprising the further step of encoding the operational data in hexadecimal format.

18. The method of claim 11, wherein the operational data received at the at least one service center comprises monitored data associated with an operational condition of the imaging system and position data associated with the location of the imaging system.

19. The method of claim 11, wherein the low earth orbit satellite system comprises a plurality of low earth orbit satellites.

20. A computer program that is stored on one of more tangible mediums for communicating data with a medical imaging system, the program comprising:
a routine for accessing monitored data associated with an imaging system;
a routine for determining a position of the imaging system from a plurality of signals; and
a routine for transmitting operational data from the imaging system to a service center via a low earth orbit satellite system.

21. The computer program, as set forth in claim 20, comprising a routine for integrating the position of the imaging system and the monitored data into the operational data for transmission to the service center.

22. The computer program, as set forth in claim 20, comprising a routine for encoding the operational data into a hexadecimal format for transmission to the service center.

23. The computer program, as set forth in claim 20, wherein the imaging system comprises a magnetic resonance imaging system.

24. The computer program, as set forth in claim 23, wherein accessing comprises polling a smart helium meter coupled to the imaging system for monitored data.

25. The computer program, as set forth in claim 20, wherein transmitting the operational data comprises communicating the operational data through a plurality of low earth orbit satellites.

26. A system for communicating data with an imaging system, the system comprising:
means for monitoring at least one imaging system for a plurality of operational conditions;
means for determining a position of at least one imaging system from a plurality of position signals; and
means for communicating the plurality operational conditions and the position to at least one service center via a low earth orbit satellite system.
